(19) United States
(12) Patent Application Publication
(10) Pub. No.: US 2006/0004292 A1
(43) Pub. Date: Jan. 5, 2006

(54) Optical examination method and apparatus particularly useful for real-time discrimination of tumors from normal tissues during surgery

(76) Inventor: Alexander Beylin, New York, NY (US)

Correspondence Address: Martin D. Moynihan;PRTSI, Inc., P.O. Box 16423, Arlington, VA, 22215, US

(21) Appl. No.: 11/149,186

(22) Filed: Jun. 10, 2005

Related U.S. Application Data
(63) Continuation-in-part of application No. PCT/IL03/01066, filed on Dec. 14, 2003.
(60) Provisional application No. 60/433,229, filed on Dec. 13, 2002.

(51) Int. Cl. A61B 6/00

(52) U.S. Cl. 600/476

(57) ABSTRACT

A method and apparatus for enhancing the optical detection of target portions of an object particularly useful in the optical examination of biological tissue to distinguish cancerous tissue from non-cancerous tissue. This is accomplished by exposing the object, e.g., biological tissue, to first and second light sources of different spectral contents for short, alternating, first and second time periods; detecting the light received from the object during each of the first and second time periods; and utilizing the light detected in the first and second time periods for producing and displaying a composite image including the image of the object and an enhanced image of the target portions, e.g., the cancerous tissue, overlayed on the image of the object.

### RELATED PATENT APPLICATIONS

This is a continuation-in-part of PCT Patent Application No. PCT/IL03/01066, filed Dec. 14, 2003, which claims the benefit of U.S. Provisional Patent Application No. 60/433,229, filed Dec. 13, 2002. The content of the above applications is incorporated herein by reference in its entirety.

### FIELD AND BACKGROUND OF THE INVENTION

The present invention relates to a method and apparatus for optically examining objects in order to more clearly discriminate target portions of interest from other portions of the object. The invention is particularly useful for the real-time discrimination of tumors from normal tissues during surgery, especially during brain neurosurgery, and is therefore described below with respect to this application, but it will be appreciated that the invention, or various features thereof, could advantageously be used also in other applications.

The prognosis of patients suffering from malignant gliomas, astrocytomas, meningiomas, and other brain tumors is strongly linked to the completeness of tumor removal. [Nitta & Sato, 1995, Devaux et al., 1993, Rostomily et al., 1994, Yoshida et al., 1994]. However, during the surgical procedure the tumor cannot always be visualized precisely. That happens often since, under the standard white light illumination used by neurosurgical microscopes, the tumors, especially their borders, are often indistinguishable from the surrounding normal brain tissue. This complicates the tumor removal procedure and makes it totally dependent on the judgment of the surgeon, which is based on his/her experience.

To localize the tumor, neurosurgeons have used diagnostic imaging methods, such as Computer Tomography (CT) or Magnetic Resonance Imaging (MRI), which enhance the contrast between tumor and surrounding brain tissue. However, the discrepancy between intraoperative observations of tumor margins and pre-operative diagnostic imaging studies has been emphasized in recent years with correlative studies of tissue obtained from regions of the brain adjacent to the imaged tumor [Green et al, 1989, Kelly, 1987, Haglund, 1994].

Ultrasound, unlike CT and MRI, has the ability to provide real-time intraoperative information to localize the tumor and to define its volume (Chandler, 1982, Enzmann, 1982, Gooding, 1984). However, the ability to localize tumor tissue by ultrasound once resection commences is limited by signal artifacts caused by blood and surgical trauma at the resection margin (LeRoux et al., Lipson et al., 1961). Thus intraoperative ultrasound is an unsatisfactory tool for assisting the neurosurgeon in efforts to completely resect primary brain tumors.

Other available methods to obtain real-time feedback during tumor resection involve the use of frozen sections or smear preparations of biopsies from the margins (Salcman, 1990). However, intraoperative biopsies are hampered by the sampling error, the wait for results, and the difficulty in distinguishing tumor cells from reactive astrocytes [Haglund et al, 1994]. Thus, this method is highly impractical.

In the last decade, relatively small open-air MRI scanners were developed for intraoperative imaging. Intraoperative MRI allows the neurosurgeon to obtain images during surgery which improve the completeness and accuracy of tumor resection. However, the intraoperative MRI scanner is not an on-line imaging system. To perform a scan, an interruption of the procedure for 10-20 minutes is required, further extending an already lengthy operation. Also, intraoperative MRI machines are bulky, obstruct and limit the surgical field, and impose special requirements on the neurosurgical facility, such as the need for a non-magnetic environment, special non-ferrous surgical instruments, etc. The cost of operating and maintaining these liquid-helium cooled machines is extremely high, and as a result, there are very few medical centers that are equipped with intraoperative MRI scanners at present.

The idea of demarcating tumors using intravenous fluorescent dyes was proposed more than fifty years ago [Moore et al, 1948]. It was found that some fluorescent markers are capable of penetrating the disrupted blood-brain barrier, and selectively labeling tumor cells. For contrasting the tumors various markers were used, such as fluorescein [Feindel et al., 1967., Boggan et al., 1984., Murray, 1982., Poon et al, 1992, Kuroiwa, 1998], and indocyanine green (ICG) [Haglund et al, 1996, Haglund et al, 1994, Hansen et al, 1993,].

However, in the operating room these methods are quite difficult to use [Cheng et al. 1990]. They are also unreliable primarily due to contamination of the surgical cavity by blood containing the fluorescent marker and leakage of dyes with accompanying edema into surrounding nontumorous tissue [Stummer et al. 1993].

Technical difficulties prevented the development of useful in vivo florescent marking systems. A partial list of such technical difficulties includes: low level of tumor fluorescence and the bright ambient light (dynamic range problem); autofluorescence of the healthy brain tissue; brain movement artifacts due to breathing and heartbeats; limited level of exposure to the excitation illumination (NIOSH and ACGIH threshold standards); and overlay of the fluorescent and white light images.

Recently, a novel method for contrasting brain tumors has been proposed. This method uses the putative capacity of malignant tissue to preferentially synthesize or accumulate fluorescent and photosensitizing endogenous porphyrins after administration of 5-aminolevulinic acid (5-ALA), a naturally occurring precursor in the heme biosynthesis pathway. When applied to human malignant gliomas, porphyrin accumulation allows fluorescence detection within the brain [Stummer et al., 1998a, Stummer et al., 1998b]. The obvious advantage of this method is that fluorescence would be restricted to malignant cells, without contamination of the tumor cavity by blood-borne marker or spreading of the marker with peri-tumoral edema.

Despite the appeal of the new tumor contrasting method, its current application remains impractical, primarily because equipment other than the microscope itself is required [Kiroiwa, 1998]. Although almost all neurosurgical operations are performed under an operating microscope, there has been no commercially available microscope with which fluorescence could be observed.

What is needed, therefore, are techniques for safely and cost effectively distinguishing between cancerous tissue and healthy tissue in real time, and an imaging system embedded into the standard operating microscope to provide real time tumor visualization of tumor fluorescence during the neurosurgical procedure. Such a system would assist neurosurgeons greatly and would contribute significantly to the field of neurosurgical oncology.

### OBJECTS AND BRIEF SUMMARY OF THE PRESENT INVENTION

An object of the present invention is to provide an improved method and apparatus for enhancing the optical detection of target portions of an object to distinguish same from other portions of the object, particularly for enhancing, in a real-time manner, the optical examination of biological tissue to distinguish cancerous tissue from non-cancerous tissue.

According to one aspect of the present invention, there is provided a method of enhancing the optical detection of target portions of an object to distinguish the target portions from other portions of the object, comprising: exposing the object to first and second sources of electromagnetic radiation of different spectral contents for short, alternating, first and second time periods; detecting the electromagnetic radiation received from the object during each of the first and second time periods; and utilizing the electromagnetic radiation detected in the first and second time periods for producing and displaying a composite image including the image of the object and an enhanced image of the target portions overlayed on the image of the object.

In the described preferred embodiments, the first source of electromagnetic radiation is a source of polychromatic light, and the second source of electromagnetic radiation is a source of monochromatic light of a particular wavelength or band of wavelengths selected such that the target portions, when exposed to the monochromatic light, return, absorb or generate more light from the target portions than from the remainder of the object.

More particularly in the described preferred embodiments, the polychromatic light is ambient light, and the monochromatic light is excitation light selected to induce fluorescence in the target portions to a greater extent than in the remainder of the object.

According to further features in the described preferred embodiments, before the object is exposed to the first and second light sources, a marker substance is administered to the object capable, when the object is exposed to the excitation light, of inducing fluorescence in the target portions of the object to a greater extent than in the remainder of the object.

According to some embodiments described below, in the first time periods the object is exposed to both the ambient light and the excitation light, and in the second time periods, alternating with the first time periods, the object is exposed only to the ambient light. The light detected in the first and second time periods is utilized for producing and displaying the composite image by subtracting the light detected in each of the second time periods from that in each of the first time periods to produce an image of only the target portions of the object; and the composite image is displayed by overlaying the image of the target portions of the object over an image of the object produced when the object is exposed to the first light source.

According to a further described preferred embodiment, in the first time periods, the object is exposed only to the ambient light, and in the second time periods, the object is exposed only to the excitation light. The time periods alternate at a high rate of at least sixty frames per second so as to eliminate or minimize flicker in the composite image displayed.

As indicated above, and as will be described more particularly below, the method of the present invention is particularly useful in surgery, especially brain neurosurgery, to distinguish cancerous tissue from non-cancerous tissue.

According to a more specific aspect of the present invention, therefore, there is provided a method of enhancing the optical examination of biological tissue to distinguish cancerous tissue from non-cancerous tissue, comprising: applying to the biological tissue a marker substance capable of inducing greater fluorescence in cancerous tissue than in non-cancerous tissue when subjected to excitation light of a predetermined wavelength or band of wavelengths; exposing the biological tissue to ambient light; exposing the biological tissue to excitation light of the predetermined wavelength or band of wavelengths such that the biological tissue is exposed during first time periods to both the excitation light and the ambient light, and during second time periods only to the ambient light; detecting light received from the biological tissue to produce: (a) a first detector output representing the light received during the first time periods when the biological tissue is exposed to both the excitation light and the ambient light, and (b) a second detector output representing the light received during the second time periods when the biological tissue is exposed only to the ambient light; subtracting one detector output from the other detector output to periodically produce differential outputs; and displaying the differential outputs as an overlay on an image of the biological tissue providing background for the cancerous tissue inducing the fluorescence.

The invention also provides apparatus for enhancing the optical detection of target portions of an object to distinguish such target portions from other portions of the object, comprising: first and second sources of electromagnetic radiation of different spectral contents; a control system for exposing the object to the first and second sources of electromagnetic radiation for short, alternating first and second time periods; an optical detector for detecting the electromagnetic radiation received from the object during each of the first and second time periods; and a processor utilizing the electromagnetic radiation detected in the first and second time periods for producing and displaying a composite image including the image of the object and an enhanced image of the target portions overlayed on the image of the object.

According to a further feature in the described preferred embodiments, the apparatus further comprises a narrow wave-band filter passing light of the predetermined wavelength or band of wavelengths, and blocking light of other wavelengths; the optical detector periodically detecting the light received from the object through the narrow waveband filter. Such an arrangement increases the signal-to-noise ratio of the displayed composite image.

According to another preferred feature in the described preferred embodiment, the apparatus further comprises a camera for producing an image of the object, the display displaying the differential outputs of the optical detector system as an overlay on the image of the object.

The described preferred embodiments are particularly useful for enhancing the optical examination of biological tissue in a real-time manner to distinguish cancerous tissue from non-cancerous tissue, in which case the excitation light source is one which induces, after a marker substance has been applied to the biological tissue, fluorescence in the predetermined wavelength, or band of wavelengths, to a greater extent in cancerous tissue than in non-cancerous tissue. Such a method and apparatus, therefore, are particularly useful in tumor resection, to provide the surgeon with images which enhance the contrast between cancerous and non-cancerous tissue and which thereby enable the surgeon to improve the completeness and accuracy of the tumor resection.

Further features and advantages of the invention will be apparent from the description below.

### BRIEF DESCRIPTION OF THE DRAWINGS

The invention is herein described, by way of example only, with reference to the accompanying drawings, wherein:

FIG. 1 is a block diagram illustrating one form of apparatus constructed in accordance with the present invention;

FIG. 2 is a timing diagram helpful in explaining the operation of the apparatus of FIG. 1;

FIGS. 3a-3f are a series of photographic prints illustrating results produced by the use of the apparatus of FIG. 1 for enhancing the optical detection of target portions of an object in accordance with the present invention;

FIGS. 4a and 4b illustrate the manner in which the enhancement technique of the present invention enhances the display of a mouse radiation-induced fibrocarcenoma (RIF) tumor, FIG. 4a illustrating the image of the tumor with surrounding tissues under ambient light, FIG. 4b illustrating the superposition of the enhanced image of the tumor and the ambient light image of the surrounding tissues;

FIG. 5 is a block diagram illustrating another apparatus constructed in accordance with the present invention embodied in a surgical microscope;

FIG. 6 illustrates a special filter technique which may be used, by means of a single imagining device, for producing and displaying both the background image and the enhanced image of the target portions overlayed thereon;

FIG. 7 is a block diagram illustrating a further apparatus constructed in accordance with the present invention embodied in a pair of head-mounted surgical loupes;

FIG. 8 illustrates the structures of the tool embedded excitation light source shown in FIG. 7;

FIG. 9 is a block diagram illustrating a still further embodiment of the invention; and

FIG. 10 illustrates the construction of a filter which used with the embodiment of FIG. 8.

It is to be understood that the foregoing drawings, and the description below, are provided primarily for purposes of facilitating understanding the conceptual aspects of the invention and various possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

As indicated earlier, the invention relates broadly to a method and apparatus for enhancing the optical detection of target portions of an object, and is particularly useful for enhancing the optical examination of biological tissue in order to distinguish cancerous tissue from non-cancerous tissue. The embodiments of the invention described below are therefore directed to the latter application, namely to enhance the optical examination of biological tissue in order to provide real-time tumor visualization during a surgical procedure, particularly a brain neurosurgical procedure, in order to better enable the surgeon to remove all the cancerous tissue with a minimum of non-cancerous tissue.

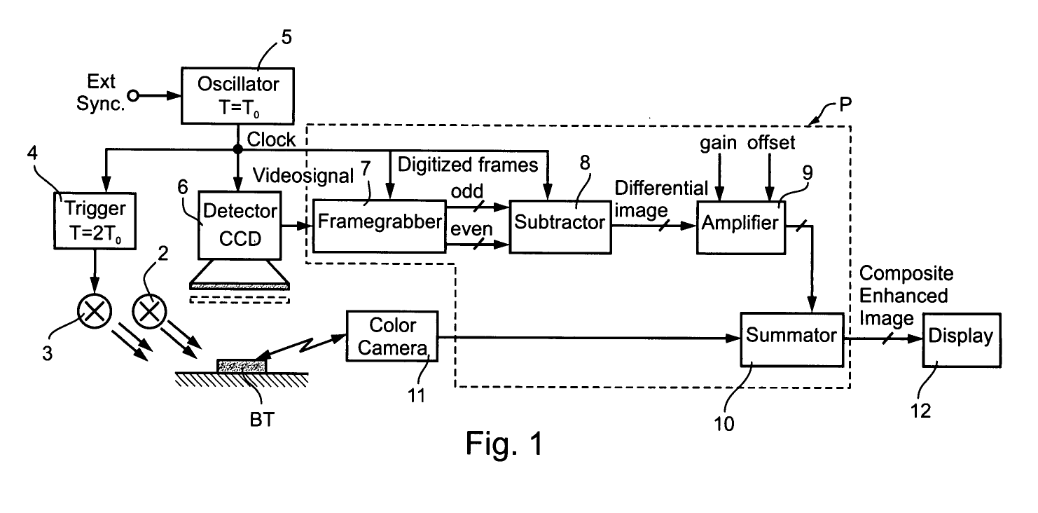

The apparatus illustrated in FIG. 1 is for use in examining biological tissue BT in a real-time manner while performing a surgical resection procedure. The biological tissue BT is continuously exposed to ambient (e.g., polychromatic or white) light, schematically indicated at 2. In addition, the biological tissue BT is periodically exposed to excitation (e.g., monochromatic) light from an excitation light source 3 of a predetermined wavelength or band of wavelengths. The excitation light source 3 is periodically activated by a trigger 4 controlled by clocking pulses from an oscillator 5 to periodically expose the biological tissue BT to the excitation light from source 3, as well as to the ambient light from source 2. It will thus be seen that during the time intervals that the excitation light source 3 is activated, the biological tissue BT receives light from both the excitation light source 3 and the ambient light source 2; whereas during the time periods that the excitation light source 3 is not activated, the biological tissue BT receives light only from the ambient light source 2.

It should be emphasized that the oscillator may generate either constant clock To or variable clock rate T(t) if synchronized with the external signals, for example, heart rate or respiratory activity, ambient light fluctuations, etc. In fact, it was found that synchronizing the clock with fluctuation of the electrical power (50/60 Hz), significantly improves the signal-to-noise ratio (S/N) of the system.

The apparatus illustrated in FIG. 1 further includes an optical detector, in the form of an imager 6, for periodically detecting the light received from the biological tissue BT as a result of being exposed to the light from sources 2 and 3. Detector 6, e.g., a CCD matrix, is controlled by clock pulses from an oscillator 5 so as to produce: (a) a first detector output representing the light received from the biological tissue BT during the time periods when the biological tissue BT is exposed to both the excitation light from source 3 and the ambient light from source 2; and (b) a second detector output representing the light received from the biological tissue BT during the other time periods when the biological tissue BT is exposed only to the ambient light from source 2.

Before the surgical operation is commenced, a marker substance is applied to the biological tissue BT. The marker substance is one capable of inducing greater fluorcence in cancerous tissue than in non-cancerous tissue when subjected to excitation light of a predetermined wavelength or band of wavelengths such as to increase the contrast between healthy brain tissue and the tumor, and thereby to assist the surgeon in performing tumor resections more completely and accurately. Images of the tumor are thus generated on-line, enabling the surgery to be performed without interruption or delay for tumor identification.

Many such marker substances are known as briefly mentioned in the above discussion of the background to the present invention. When the apparatus illustrated in FIG. 1 is used for tumor identification during brain surgery, the preferred method for contrasting brain tissues relies on the observation that malignant tissue preferentially synthesizes or accumulates fluorescent and photosensitizing endogenous porphyrins, such as 5-aminolevulinic acid (5-ALA), a naturally occurring precursor in the heme biosynthesis pathway. When applied to human malignant gliomas, porphyrin accumulation allows fluorescence detection within the brain. The obvious advantage of this method is that fluorescence would be restricted to malignant cells, without contamination of the tumor cavity by blood-borne marker or spreading of the marker with peri-tumoral edema.

Such a marker substance is capable of inducing greater fluorescence in cancerous tissue than in non-cancerous tissue when subjected to excitation light in the 395 nm spectral range. Accordingly, the excitation light source 3 would have a wavelength or band of wavelengths within this spectral range.

The apparatus illustrated in FIG. 1 further includes a processor for periodically subtracting one detector output from the other detector output to periodically produce differential outputs. Such a processor is schematically indicated by broken lines P in FIG. 1 as including a framegrabber 7 and subtractor 8. Framegrabber 7 digitizes the above two outputs of detector 6. Subtractor 8 digitally subtracts one output from the other to periodically produce differential outputs. Since these differential outputs represent the light received from the biological tissue BT when exposed to both light sources 2 and 3, but after the ambient light from light source 2 has been subtracted, such differential outputs represent largely the fluorescence in the cancerous tissue resulting from the administration of the marker substance.

According to one embodiment of the invention, detector 6 is a PixelFly, monochrome, 12 bit digital camera manufactured by PCO Computer Optics GmbH, Germany, and distributed by Cooke Corp, Auburn Hills, Mich. This camera which has a higher dynamic range provides 40 frames/second frame rate and features a specific mode of operation called the “double shot” mode. In this mode, the camera acquires images by series of two frames. The exposure of the second frame is synchronized with the readout of the first frame. As a result, there is virtually no gap between two successive frames in series. If exposure time of each frame is short enough, the movement artifact is small.

According to this embodiment, the camera (detector 6) generates the series of frame pairs such as to have a short time interval between the frames of each pair, and a longer time interval between the pairs of frames. In the described preferred embodiment, the gap between successive frames in each pair is less than 7 us; and the selected exposure time for each frame is 6.8 ms.

It will thus be seen that, in FIG. 1, framegrabber 7 outputs a sequence of “odd” digitized frames, e.g., representing the light received from the biological tissue BT when exposed to both the excitation light from source 3 and the ambient light from source 2, and “even” digitized frames, e.g., representing the light received from biological tissue BT only when exposed to the ambient light from source 2. Framegrabber 7 digitizes the image of each frame to produce a sequence of odd frames alternating with even frames. Subtractor 8 digitally subtracts the detector output of each frame of the sequence from that of the preceding frame of the sequence, to thereby produce a differential output largely representing the light received by the fluorecence of the cancerous tissue.

The differential outputs from subtractor 8 are amplified in amplifier 9 which is controlled in a conventional manner according to the desired gain and offset, before feeding the output to summator 10. This information received by summator 10 thus represents the light received by fluorecence from the cancerous tissue.

When the apparatus is used during a surgical procedure to image the cancerous tissue in a real-time manner, it is highly desirable to overlay this image on a background image of the other tissue in the surgical site. For this reason, FIG. 1 includes a color camera 11 oriented to image the biological tissue BT at the surgical site and to produce an electrical output to summator 10 representing that image. The output received by summator 10 from amplifier 9, representing the cancerous tissue, is thus overlayed on the background image from color camera 11, to thereby produce a composite image in display 12 of the surgical site in which the image of the cancerous tissue has been enhanced in the above-described manner in order to better distinguish such tissue from non-cancerous tissue.

To increase the signal-to-noise ratio for the fluorescent signal, a narrow bandpass barrier filter 13 (633FS10-50, Andover Corp.), was used, attached to the detector 6 lens. Filter 13 was tuned for the emission peak of PpIX (632 nm) and effectively attenuates all other wavelengths. Also, framegrabber 7 and camera 11 may be separated by a wireless link

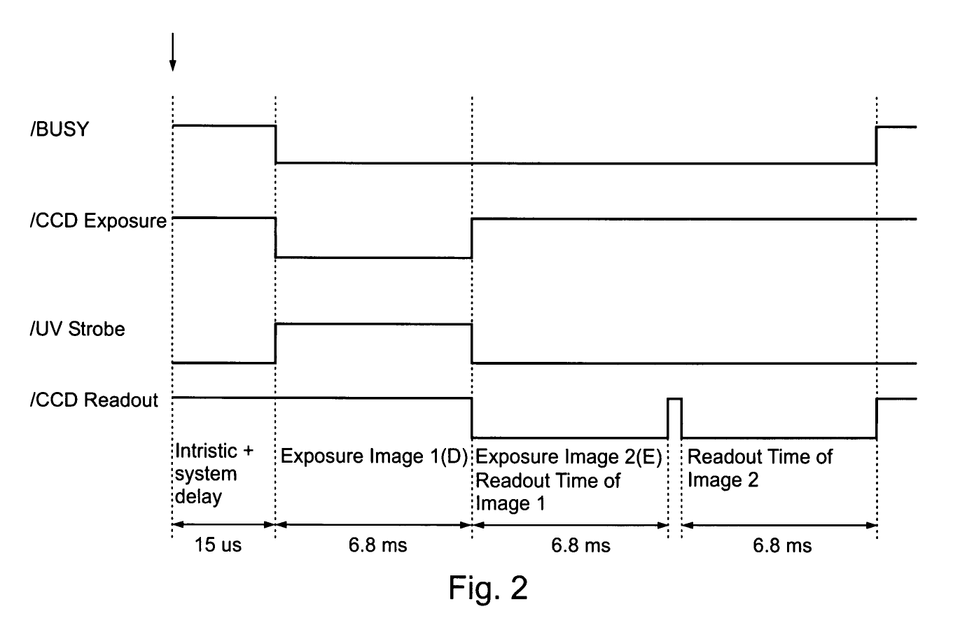

FIG. 2 illustrates timing diagrams when the excitation light source 3 is an excitation illumination strobe and the detector 6 is a camera including a CCD matrix operating according to the above-described “double-shot” mode. For generating the excitation illumination strobe, the light source is controlled by the CCD exposure signal generated by the camera 6. Since the CCD exposure signal extends only for the duration of the first frame, the ultraviolet light goes off during the second frame exposure. The differential image is calculated by framegrabber 7 and subtractor 8 in the above described manner. The latter may be in a host computer, e.g., Pentium III/600 mhz, 512 Mb ram.

In a test of the above-described embodiment of the invention, a fluorescent signal was registered from a vial of protoporphyrin IX dissolved in dimethyl sulfoxide (DMSO) at a concentration of 0.9 nM/ml, which is considerably lower than the concentration of PpIX found in tumor cells [Abels et al., 1997]. A control vial was filled with pure DMSO. The excitation light sources included a cluster of 6 LEDs (in one embodiment L200CUV395-12D, Ledtronics Inc.) located 120 mm from the imaging object, and delivered light in the 395 nm spectral range. The ambient light, represented by light source 2, was the normal room lighting (white light).

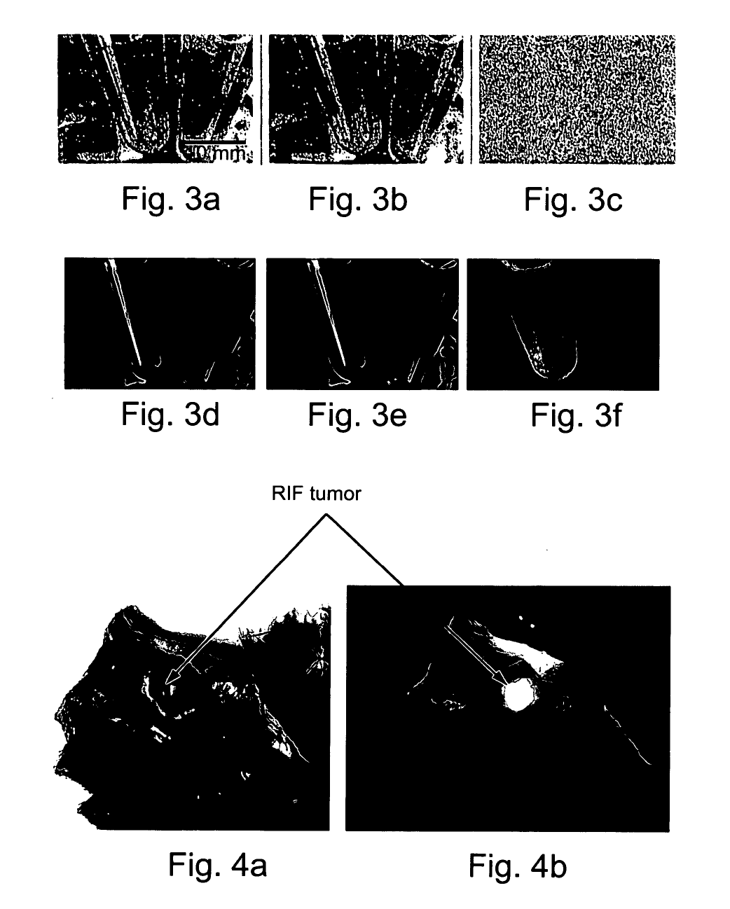

The results of this test are illustrated in FIGS. 3a-3f showing the tips of two 1.5 ml vials laying on a glossy background. The left vial was filled with PpIX solution in DMSO; whereas the right vial was filled with saline and used as a control.

In the upper row, FIGS. 3a and 3b picture the two vials when exposed only to ambient light, i.e., without the excitation strobe light; whereas FIG. 3c represents the differential image produced by subtracting the image of FIG. 3b from that of FIG. 3a. It will be seen that FIG. 3c thus includes no image, but only noise.

In the lower row, FIG. 3d represents the image produced when both vials were subjected to both ambient light and the excitation illumination strobe; whereas FIG. 3e represents the image when the two vials were exposed only to the ambient light. FIG. 3f pictures the differential image produced when the image of FIG. 3e is subtracted from that of FIG. 3d. It will be seen that the differential image of FIG. 3f thus shows only the left vial of FIGS. 3d and 3e, namely the one containing the PpIX solution in DMSO.

FIGS. 4a and 4b demonstrate the above-described image-enhancement technique when applied to imagining a tumor of a mouse (RIF tumor). Thus, FIG. 4a is an image of the tumor with the surrounding tissues under ambient light; whereas FIG. 4b illustrates the super position of the enhanced image of the tumor as an overlay on the ambient light image of the surrounding tissues.

From the above, it will be seen that the double-shot 12 bit PixelFly camera provides clear differential image of PpIX fluorescence, with concentration similar to the real brain tumors. Moreover, the differential images are robust to the relatively slow movements of the images and can be calculated with 20 frames/second sustain rate on mid-priced PC.

Although the exposure time of the first frame can be controlled by the user in the range from 10 us to 10 ms, the exposure time of the second frame solely depends on the vertical binning selection as shown in Table 1:

TABLE 1

| | |
|---|---|
| V1 binning | 24.8 ± 0.5 ms |
| V2 binning | 12.8 ± 0.5 ms |
| V4 binning | 6.8 ± 0.5 ms |

For differential image calculation, it is very important to balance the background brightness in both images. Therefore, when acquiring full resolution images with exposure time (e.g., of 10 ms) an external shutter for the second frame should be used, e.g., liquid crystal electronic shutter. As an alternative, a fast CCD camera may be used.

It will thus be seen that the above-described method and apparatus, when used with a marker substance inducing fluorescence in cancerous tissue, can serve as a Neurosurgical Imaging Enhancement System (NIES) particularly in neurosurgical oncology to increase the contrast between healthy brain tissue and tumor, and thereby to greatly assist the neurosurgeon to perform tumor resections more completely and accurately. Since the images of the tumor are generated on-line, this enables the surgery to be performed without interruption or delay for tumor identification.

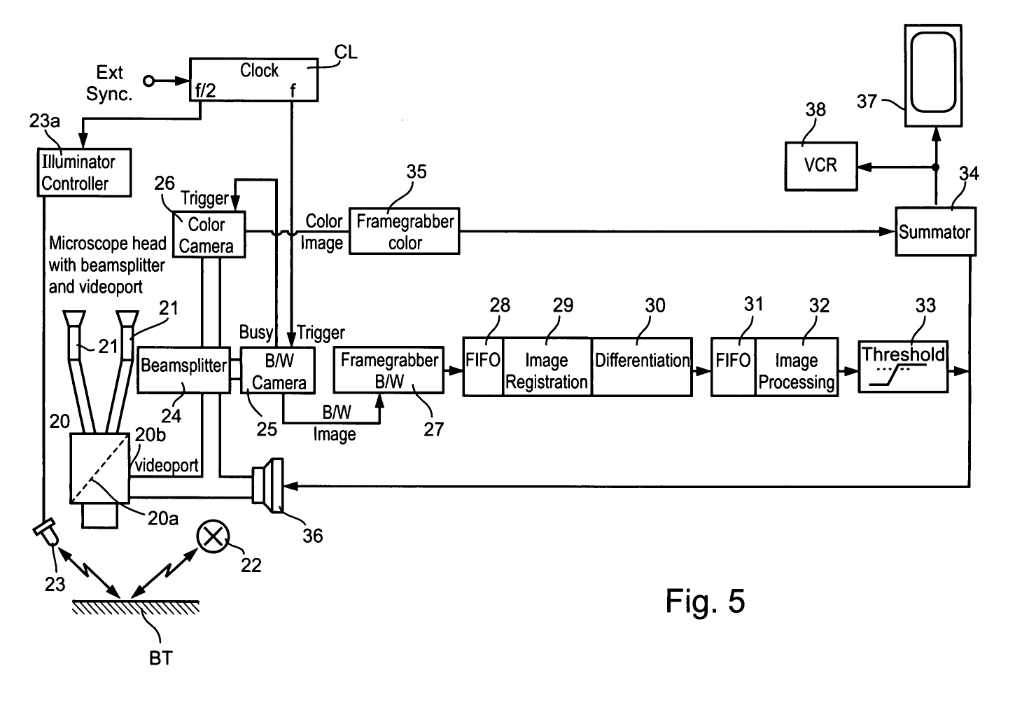

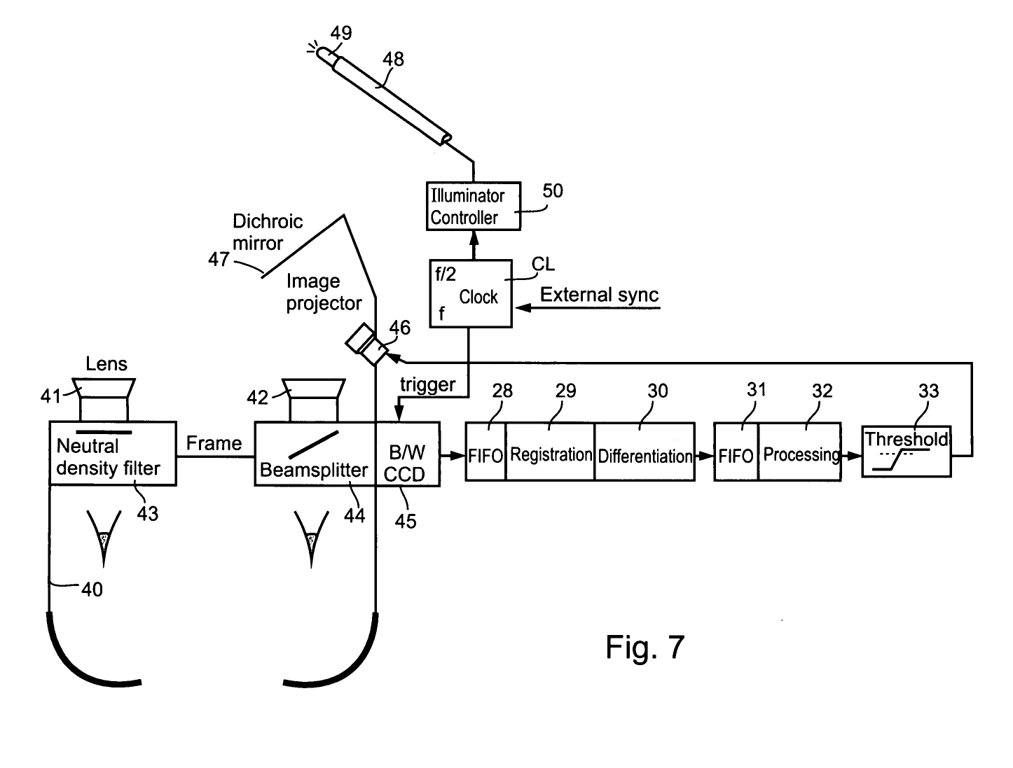

The system would preferably be used with a surgical visual instrument, such as a surgical microscope or head-mounted surgical loupes, to enable the surgeon to view the enhanced image of the cancerous tissue overlayed on the background tissue at the surgical site. FIG. 5 is a block diagram illustrating the above-described Neurosurgical Imaging Enhancement System (NIES) incorporated in a surgical microscope; whereas FIG. 7 illustrates the system incorporated in a head-mounted pair of surgical loupes.

With reference to FIG. 5 illustrating a surgical microscope implementation of the invention, the surgical microscope includes a microscope head 20 and a pair of eye-pieces 21 for viewing the biological tissue BT. The biological tissue is exposed both to ambient (polychromatic) light from ambient light source 22, such as the conventional microscope illuminator, surgical overhead lamps, room lights, etc, and to excitation (monochromatic) light from excitation light source 23 of a selected predetermined wavelength or band of wavelengths.

The excitation light source 23 is controlled by a controller 23a, which is turn controlled by clocking pulses from clock CL, to periodically activate the excitation light source 23 in the manner described above with respect to FIG. 1. Thus, the biological tissue BT is exposed, during first time periods, to both the excitation (monochromatic) light from source 23 and the ambient (polychromatic or white) light from source 22, and during other time periods, only to the ambient light from light source 22.

Microscope head 20 includes a beam splitter 20a allowing viewing the biological tissue BT and also producing an optical output via a video port 20b to the camera beamsplitter 24. The latter produces one video output to a B/W camera 25, and another output to a color camera 26. The B/W camera 25 corresponds to detector 6 in FIG. 1 for enhancing the fluorescent image of the cancerous cells; whereas color camera 26 corresponds to camera 11 in FIG. 1 to produce the background image over which the enhanced image of the cancer tissue is overlayed.

Thus, the B/W camera 25 produces the two detector outputs described above in FIG. 1, one detector output representing the light received during the time periods when the biological tissue is exposed to both the exciting light and the ambient light, and the other detector output representing the light received during the other time periods when the biological tissue is exposed only to the ambient light. These outputs are fed to a processor, wherein they are digitized by a B/W framegrabber 27, stored in a FIFO memory 28, aligned in an image registration module 29, and differentiated in a differentiation module 30 by periodically subtracting one detector output from the other detector output, to periodically produce differential outputs. The differential outputs are fed to another FIFO memory 31, image processing module 32, and threshold module 33. The latter suppresses the signals below a predetermined threshold before the signals are applied to summator 34, corresponding to summator 10 in FIG. 1, which overlays the enhanced image of the cancerous tissue over the background image derived from color camera 26.

Thus, the operation of color camera 26 is synchronized by trigger pulses from clock CLwith the operation of the B/W camera 25. The color image is digitized in color camera 26 to a digital output and is fed to another framegrabber 35 before being fed to the summator 34 to provide the background for the composite image produced by the summator, namely the background from color camera 26 overlayed with the enhanced image of the cancerous tissue produced by the B/W camera 25.

The digital data representing the composite image produced in the summator 34 is fed to an image injection micro display unit 36, which converts the digital signal into an optical image fed to the microscope head 20 to enable its viewing via the beam splitter 20a and the eye pieces 21.

It will thus be seen that the apparatus illustrated in FIG. 5 provides the surgeon in real time with a view of the surgical site having an increased contrast between the cancerous tissue and the non-cancerous tissue, thereby greatly assisting the surgeon to perform tumor resections more completely and accurately.

As shown in FIG. 5, the composite image from summator 34 may also be fed to an external monitor 37 for viewing by surgical assistants, and/or to a video recorder 38 for archiving and storage purposes.

In FIG. 7, illustrating the invention embodied in surgical loupes, the apparatus includes a frame 40 for head-mounting a pair of lens units 41, 42 in alignment with the surgeon's eyes. One lens unit 42 houses the image-enhancement apparatus described above, e.g., the apparatus similar to one described in FIG. 5, without the color camera, the color frame grabber and the summator module. The other lens unit 41 houses a neutral density filter 43 for reducing the light intensity of the light viewed via lens unit 41 so as to be substantially equal to the light intensity viewed via lens 42 because of the reduction in the light intensity in the latter lens unit resulting from splitting the image to the image enhancement system.

The image enhancement system acting on the lens unit 41 includes a beam splitter unit 44 corresponding to that in the microscope head 20 in FIG. 5 for receiving the light via the lens unit 41; a B/W CCD imager 45; and an image enhancement system corresponding to elements 28-33 illustrated in the system of FIG. 5 between the beam splitter 24 and the image injection micro display 36; and an image injection micro display 46, corresponding to micro display 36 in FIG. 5. Thus, the system in FIG. 7 functions in the same manner to produce a digital output of the enhanced cancerous tissue. The image injection micro display 46 converts the digital form of this image to video, which video is projected on a dichroic mirror 47 through which the surgical site is viewed by the surgeon via lens unit 41; therefore, the color background image of the surgical site and the enhanced image of the cancerous tissue are superimposed optically.

In the microscope implementation of the invention, as illustrated in FIG. 5, it will be seen that the excitation light source 23 is mounted on the microscope and delivers the light to the object BT via a path coaxial with the image path of the microscope, this being permitted by the beam splitter 20a. When the invention is implemented in head-mounted surgical loupes as illustrated in FIG. 7, the excitation light would normally be mounted on the remote tip of a surgical instrument, as schematically shown in FIG. 7 by surgical instrument 48 carrying, at its tip, the excitation light source 49 controlled by illuminator controller 50 via the clock CL. Such a surgical instrument may be, for example, a suction tube, forceps, or the like.

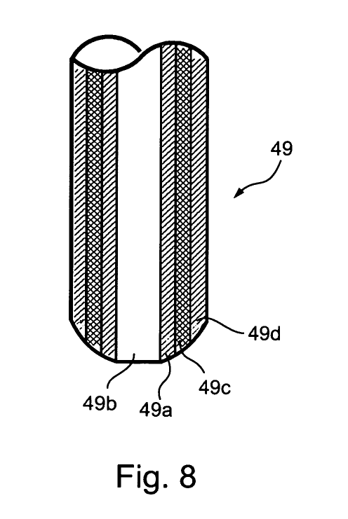

FIG. 8 illustrates an example of the construction of a surgical suction tube with an embedded light source 49, wherein it will be seen it includes a suction tube 49a defining an inner lumen 49b, an optical fiber layer 49c for conducting the light from a light source (not shown), and an outer steel tube 49d to which the optical fiber layer may be bonded.

In the microscope set up illustrated in FIG. 5, the cameras 25 and 26, and the image injecting micro display 36, are aligned with the same optical path of either the left eye or right eye to avoid optical parallax. Similarly, identical adapters can be installed on both sides of the microscope in order to produce true 3D contrast enhanced images.

While the above-described embodiments illustrate a dual-imager set-up, in which one imager (camera) is used for registration of fluorescence, and the second for registration of background, the invention may also be embodied in single imager setups in which both the background (color) and the fluorescence image (monochrome) are captured by the same imager. When using such a setup, the imager, e.g., a CCD matrix would include a special filter mask, wherein the standard RGB mask in a color camera is interleaved with an E-pixels emission filter mask, as set forth, for example, in the following Table 2:

TABLE 2

| | | | | | | |
|---|---|---|---|---|---|---|
| G | E | B | E | G | E | B |
| R | E | G | E | R | E | G |
| G | E | B | E | G | E | B |
| R | E | G | E | R | E | G |

That is, filter mask assembly would be attached in front of the imaging device, and would include an RGB color filter mask interleaved with an emission (E) filter mask for separating and displaying the output of the imagining device into a color image of the object from RGB pixels, and an enhanced image of the target portions from E pixels overlayed on the image of the object.

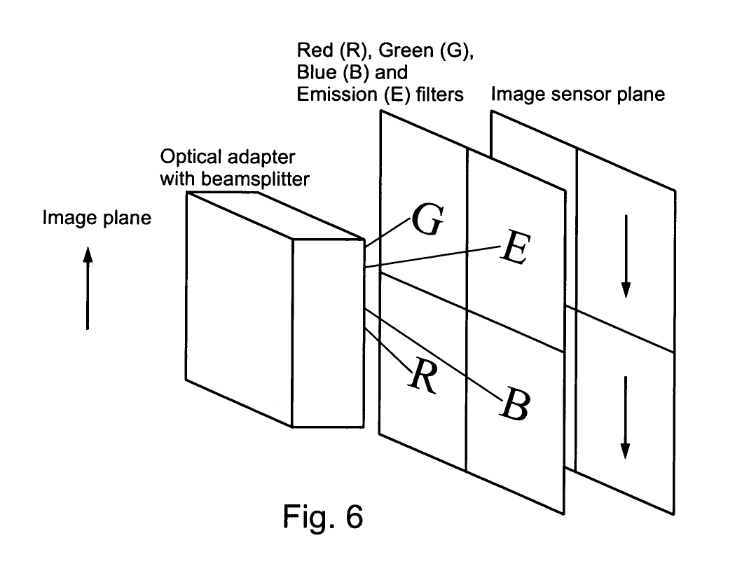

Another single-imager layout may be provided as illustrated in FIG. 6 also including a special filter, in which the area of the imager is divided into four or more quadrants. The image is optically split and projected on the specific quadrant passed through the designated filter. An example of such a filter layout is shown in Table 3:

TABLE 3

| | |
|---|---|
| R | G |
| B | E |

In such a configuration, the optical detector would include an imagining device for imagining the object, a beamsplitter, for splitting the image of the object into at least four identical images and projecting the images onto four spatially separated quadrants of the optical sensor, and a special filter installed in front of the imagining device. The area of the imagining device would be divided into at least four quadrants, one for each of the three colors RGB, and the fourth for the emission light. The RGB quadrants of the imaging device would be used for synthesis of color image of the object, and the E quadrant of the imaging device would be used for displaying the enhanced image of the target portions.

A single imager layout may be implemented on a triple sensor camera or on a camera employed a single sensor with a standard color mask. In that case, the difference signal may be derived from the group of pixels masked by the filter(s) with the maximum transmission at the fluorescence wavelength

In the single sensor color camera, the difference images may be calculated separately between the “red”, “green” or “blue” pixels of the sensor. For example, if the image includes the component of protoporhyrine-IX fluorescence at 635 nm, the bulk of the fluorescent signal is observed by“red” pixels of the sensor.

In 3-sensor (3CMOS, 3CCD) cameras, the separate difference images may be calculated from the outputs of the individual sensors. In the same example of the Protoporhyrine IX fluorescence, the difference image can be derived from the output of the “red” sensor.

The apparatus may include additional features. For example, the apparatus could include a notch filter tuned to the emission wavelength, which filter is introduced into the illumination pathway of the, e.g., microscope. In addition, the apparatus could include a shutter, electronic or mechanical, introduced into the illumination pathway for briefly blocking the light while the monochrome CCD is exposed. Further, a light chopper or other shutter arrangement could be used for periodically exposing the object to the excitation light.

For delivering the excitation light, there can be used an array of LEDs, lasers, or Xenon lights. The excitation light source may be operated continuously, while the biological tissue, or other object, is exposed periodically via a light chopper or other form of controlled shutter.

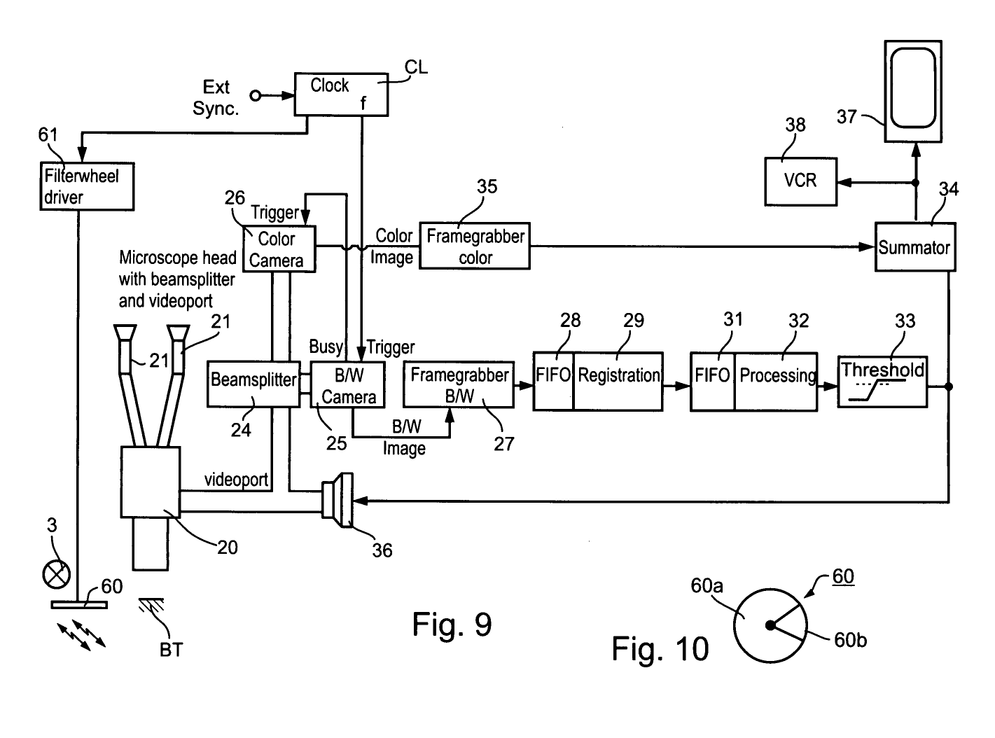

FIG. 9 is a block diagram illustrating apparatus of substantially the same construction as that illustrated in FIG. 5, except that the apparatus in FIG. 9 does not produce a differential image of the light detected from the two light sources in the first and second time periods. Rather, the apparatus illustrated in FIG. 9 exposes the object only to ambient light in the first time periods, and only to excitation light in the second time periods, the two time periods alternating at a high rate, preferably at least 60 frames per second, so as to eliminate or minimize flicker in the composite image displayed. Thus, the surgeon will see the enhanced image of the target portions produced by the excitation light during the second time periods overlayed on the image of the object produced by the ambient light during the first time periods.

Accordingly, in the apparatus illustrated in FIG. 9, there is no differentiation module, corresponding to module 30 in FIG. 5. The apparatus in FIG. 9 is therefore identified by the same reference numerals as in FIG. 5, but omitting the differentiation module 30. Instead, the apparatus of FIG. 9 would be controlled to expose the object only to the ambient light during the first time periods, and only to the excitation light 3 during the second time periods, and to alternate these exposures at a rate of at least twenty frames per second so as to eliminate or minimize flicker in the composite image displayed in display 12. This is done, in the embodiment illustrated in FIG. 9, by a filter wheel 60, between an external light source 3 driven by a driver 61 controlled by clock CL. As shown in FIG. 10, filter wheel 60 is made of transparent glass to transmit the ambient light through, a section 60a thereof, and includes a bandpass excitation filter section 60b to transmit the excitation light.

While the invention has been described particularly with respect to enhancing the optical examination of biological tissue to distinguish cancerous tissue from non-cancerous tissue, it will be appreciated that the invention could also advantageously be used in other applications for enhancing the optical detection of target portions of an object to distinguish such target portions from other portions of the object. The excitation light may not only be light which generates fluorescence, but also light which is reflected from, or absorbed by, the target portions of the object to a greater extent than in the remainder of the object. An example of a non-medical application would be in a stroboscopic system for reducing or eliminating motion of moving elements by using a stroboscopic excitation light source of a predetermined wavelength or band of wavelengths capable of being reflected, absorbed, or inducing fluorescence, from the elements of interest to a larger extent than from the remainder of the object.

Further features and applications of the invention will be apparent to those skilled in the art.

### REFERENCES

1. Abels, C., Fritisch, C., Bolsen, K., Szeimies, R.-M., Ruzicka, T., Goerz, G. Goetz, A. E. (1997) Photodynamic therapy with 5-aminolaevulinic acid-induced porphyrins of an amelanotic melanoma in vivo, J of Photochemistry and Photobiology; 40: 76-83.

2. Boggan J E, Walter R, Edwards M S B, Borcich J K, Davis R L, Koonce M, Berns M W. (1984) Distribution of hematoporphyrin derivative in the rat 9L gliosarcoma brain tumor analyzed by digital video fluorescence microscopy. J Neurosurg; 61:1113-1119.

3. Chandler W F, Knake J E, McGillicuddy J E, Lillehei K O, Silver T M (1982) Intraoperative use of real-time ultrasonography in neurosurgery. J Neurosurg 57:157-163.

4. Cheng M T, McKean J, Boisvert D, Tulip J (1986) Photoradiation therapy: Current status and applications in the treatment of brain tumors. Surg Neurol 25:423-435.

5. Devaux B C, O'Fallon J R, Kelly P J (1993) Resection, biopsy, and survival in malignant glial neoplasms. A retrospective study of clinical parameters, therapy and outcome. J Neurosurg 78: 767-775.

6. Enzmann D R, Wheat R, Marshall W H, Bird R, Murphy-Irwin K, Karbon K, Hanbery J, Silverberg G D, Britt R H, Shuer L (1985) Tumors of the central nervous system studied by computed tomography and ultrasound. Radiology 154:393-399.

7. Feindel W, Yamamoto Y L, Hodge C P. (1967) Intracarotid fluorescein angiography: a new method for examination of the epicerebral circulation in man. Can Med Ass J, 96:1-7.

8. Gooding G A W, Boggan J E, Weinstein P R: (1984) Characterization of intracranial neoplasms by CT and intraoperative sonography. AJNR Am J Neuroradiol 5:517-520.

9. Haglund M M, Berger M S, and Hochman D W. (1996) Enhanced optical imaging of human gliomas and tumor margins. Neurosurgery; 38:308-17.

10. Haglund M M, Hochman D W, Spence A M, and Berger M S. (1994) Enhanced optical imaging of rat gliomas and tumor margins. Neurosurgery; 35: 930-41.

11. Hansen D A, Spence A M, Carski T, and Berger M S. (1993) Indo-cyanine green (ICG) staining and demarcation of tumor margins in a rat glioma model. Surg Neurol; 40:451-6.

12. Kelly P J, Daumas-Duport C, Kispert D S, Kall B A, Scheithauer B W, Illig J J (1987) Image-guided stereotaxic serial biopsies in untreated glial neoplasms. J Neurosurg 66:865-874.

13. Kuroiwa T, Kajimoto Y, and Ohta T. (1998) Development of a fluorescein operative microscope for use during malignant glioma surgery. A technical note and preliminary report. Surg Neurol., 50:41-49.

14. LeRoux P D, Berger M S, Ojemann G A, Wang K, Mack L A (1989) Correlation of intraoperative ultrasound tumor volumes and margins with preoperative computerized tomography. J Neurosurg 71:691-698.

15. Lipson R L, Baldes E J, Olsen A M (1961) Hematoporphyrin derivative: A new aid for endoscopic detection of malignant disease. J Thorac Cardiovasc Surg 42:623-629.

16. Moore G E, Peyton W T, French L A, and Walker W W. (1948) The clinical use of fluorescein in neurosurgery. The localization of brain tumors. J Neurosurg 5:392-8.

17. Murray K J. (1982) Improved surgical resection of human brain tumors: Part 1. A preliminary study. Surg Neurol; 17:316 -9.

18. Nitta T, Sato K (1995) Prognostic implications of the extent of surgical resection in patients with intracranial malignant gliomas. Cancer 75:2727-2731.

19. Poon W S, Schomacker K T, Deutsch T F, Martuza R L. (1982) Laser-induced fluorescence: experimental intraoper-ative delineation of tumor resection margins. J Neurosurg;76:679-686.

20. Rostomily R C, Spence A M, Duong D, McCormick K, Bland M, Berger M S (1994) Multimodality management of recurrent adult malignant glioma: results of a phase II multiagent chemotherapy study and analysis of cytoreductive surgery. Neurosurgery 35: 378-388.

21. Salcman M: Malignant glioma management. Neurosurg Clin N Am 1: 49-63, 1990.

22. Stummer W, Götz C, Hassan A, Heimann A, Kempski O (1993) Kinetics of Photofrin II in perifocal brain edema. Neurosurgery 33:1075-1082.

23. Stummer, W., Stepp H., Möller, G., Ehrhardt A., Leonhard, M., and Reulen, H. J. (1998) Technical Principles for Protoporphyrin-IX-Fluorescence Guided Microsurgical Resection of Malignant Glioma Tissue. Acta Neurochir. (Wien) 140: 995-1000.

24. Stummer, W., Stocker, S., Wagner, S., Stepp, H., Fritsch, C., Goetz, C., Goetz, A., Kiefmann, R., and Reulen, H. J. (1998) Intraoperative Detection of Malignant Gliomas by 5-Aminolevulinic Acid-induced Porphyrin Fluorescence. Neurosurgery 42:518-526.

25. Stummer, W., Novotny, A., Stepp, H., Goetz, C., Bise, K., and Reulen H. J. (2000) Fluorescence-guided resection of glioblastoma multiforme by using 5-aminolevulinic acid-induced porphyrins: a prospective study in 52 consecutive patients. J Neurosurg 93:1003-1013.

26. Yoshida J, Kajita Y, Wakabayashi T, Sugita K (1994). Long-term follow-up results of 175 patients with malignant glioma: importance of radical tumor resection and postoperative therapy with interferon, ACNU and radiation. Acta Neurochir. (Wien) 127: 55-59.

What is claimed is:

1. A method of enhancing the optical detection of target portions of an object to distinguish the target portions from other portions of the object, comprising:
exposing the object to first and second sources of electromagnetic radiation of different spectral contents for short, alternating first and second time periods;
detecting the electromagnetic radiation received from said object during each of said first and second time periods;
and utilizing the electromagnetic radiation detected in said first and second time periods for producing and displaying a composite image including the image of the object and an enhanced image of said target portions overlayed on the image of the object.

2. The method according to claim 1, wherein said first source of electromagnetic radiation is a source of polychromatic light, and said second source of electromagnetic radiation is a source of monochromatic light of a particular wavelength or band of wavelengths selected such that said target portions, when exposed to the monochromatic light, return, absorb or generate more light from said target portions than from the remainder of said object.

3. The method according to claim 2, wherein said polychromatic light is ambient light, and said monochromatic light is excitation light selected to induce fluorescence in said target portions to a greater extent than in the remainder of the object.

4. The method according to claim 3, wherein, before said object is exposed to said first and second light sources, a marker substance is administered to said object capable, when the object is exposed to said excitation light, of inducing fluorescence in said target portions of the object to a greater extent than in the remainder of the object.

5. The method according to claim 3, wherein said object is biological tissue, and said monochromatic light is selected to induce fluorescence in cancerous tissues to a greater extent than in non-cancerous tissue.

6. The method according to claim 3, wherein, in said first time periods, the object is exposed to both the ambient light and the excitation light, and in said second time periods, alternating with said first time periods, the object is exposed only to the ambient light;
and wherein the light detected in said first and second time periods is utilized for producing and displaying said composite image by subtracting the light detected in each of said second time periods from that in each of said first time periods to produce an image of only the target portions of the object; and overlaying the image of the target portions of the object over an image of the object produced when the object is exposed to said first light source.

7. The method according to claim 3, wherein in said first time periods, the object is exposed only to said ambient light; and in said second time periods, the object is exposed only to said excitation light.

8. The method according to claim 7, wherein said time periods alternate at a high rate so as to eliminate or minimize flicker in the composite image displayed.

9. The method according to claim 3, wherein said composite image is displayed for viewing through a surgical visual instrument.

10. The method according to claim 9, wherein said surgical visual instrument is a surgical microscope.

11. The method according to claim 9, wherein said surgical visual instrument is a head-mounted surgical loupe.

12. A method of enhancing the optical examination of biological tissue to distinguish cancerous tissue from non-cancerous tissue, comprising:
applying to said biological tissue a marker substance capable of inducing greater fluorescence in cancerous tissue than in non-cancerous tissue when subjected to excitation light of a predetermined wavelength or band of wavelengths;
exposing the biological tissue to ambient light;
exposing the biological tissue to excitation light of said predetermined wavelength or band of wavelengths such that said biological tissue is exposed during first time periods to both the excitation light and the ambient light, and during second time periods only to the ambient light;
detecting light received from said biological tissue to produce:
(a) a first detector output representing the light received during the first time periods when the biological tissue is exposed to both the excitation light and the ambient light, and
(b) a second detector output representing the light received during the second time periods when the biological tissue is exposed only to the ambient light;
subtracting one detector output from the other detector output to produce differential outputs;
and displaying said differential outputs as an overlay on an image of the biological tissue providing background for the cancerous tissue inducing the fluorescence.

13. The method according to claim 12, wherein said first and second time periods alternate and are of substantially the same time duration.

14. The method according to claim 12, wherein said differential outputs are viewed through a surgical visual instrument.

15. The method according to claim 14, wherein said surgical visual instrument is a surgical microscope.

16. The method according to claim 14, wherein said surgical visual instrument is a head-mounted surgical loupe.

17. Apparatus for enhancing the optical detection of target portions of an object to distinguish the target portions from other portions of the object, comprising:
first and second sources of electromagnetic radiation of different spectral contents;
a control system for exposing the object to said first and second sources of electromagnetic radiation for short, alternating, first and second time periods;
an optical detector for detecting the electromagnetic radiation received from said object during each of said first and second time periods;
and a processor utilizing the electromagnetic radiation detected in said first and second time periods for producing and displaying a composite image including the image of the object and an enhanced image of said target portions overlayed on the image of the object.

18. The apparatus according to claim 17, wherein said first source of electromagnetic radiation is a source of polychromatic light, and said second source of electromagnetic radiation is a source of monochromatic light of a particular wavelength or band of wavelengths selected such that said target portions, when exposed to the monochromatic light, return, absorb or generate more light from said target portions than from the remainder of said object.

19. The apparatus according to claim 18, wherein said polychromatic light is ambient light, and said monochromatic light is excitation light selected to induce fluorescence in said target portions to a greater extent than in the remainder of the object.

20. The apparatus according to claim 19, wherein said object is biological tissue, and said monochromatic light is selected to induce fluorescence in cancerous tissues to a greater extent than in non-cancerous tissue.

21. The apparatus according to claim 19, wherein said control system exposes the object during said first time periods to both the ambient light and the excitation light, and in said second time periods, alternating with said first time periods, to only the ambient light;
and wherein said processor utilizes the light detected in said first and second time periods for producing and displaying said composite image by subtracting light detected in each of said second time periods from that of each of said first time periods to produce an image of only the target portions of the object, and overlaying the image of the target portions of the object over an image of the object produced when the object is exposed to said first light source.

22. The apparatus according to claim 16, wherein said processor system for periodically subtracting one detector output from the other detector output to periodically produce differential outputs comprises:
a framegrabber which digitizes the image of each frame to produce a sequence of odd frames during the first time period alternating with a sequence of even frames during the second time periods;
and a subtractor for digitally subtracting the detector output of each frame of the sequence from that of the preceding frame of the sequence.

23. The apparatus according to claim 19, wherein said apparatus further comprises a narrow wave-band filter passing light of said predetermined wavelength or band of wavelengths, and blocking light of other wavelengths; said optical detector periodically detecting the light received from said object through said narrow waveband filter.

24. The apparatus according to claim 23, wherein said apparatus further comprises a camera for producing an image of said object, said display displaying said differential outputs of the optical detector system as an overlay on the image of said object.

25. The apparatus according to claim 24, wherein said excitation light source is one which induces, after a marker substance has been applied to biological tissue, fluorescence of said predetermined wavelength, or band of wavelengths, to a greater extent in cancerous tissue than in non-cancerous tissue, thereby enabling the apparatus to be particularly useful for the examination of biological tissue in order to distinguish cancerous tissue from non-cancerous tissue.

26. The apparatus according to claim 19, wherein said apparatus further comprises a surgical visual instrument for viewing said composite image.

27. The apparatus according to claim 26, wherein said surgical visual instrument is a surgical microscope.

28. The apparatus according to claim 27, wherein said excitation light is mounted on the microscope and delivers the light to said object via a path coaxial with the image path of the microscope.

29. The apparatus according to claim 26, wherein said surgical visual instrument is a head-mounted surgical loupe.

30. The apparatus according to claim 29, wherein said excitation light is mounted on the remote tip of a surgical instrument.

31. The apparatus according to claim 19, wherein said optical detector includes a camera operating according to a “double-shot” mode to generate images of the object in a series of frame pairs, one frame of each pair imaging the object during first time periods when exposed to both the excitation light and the ambient light, the other frame of the pair imaging the object during the second time periods when exposed only to the ambient light.

32. The apparatus according to claim 31, wherein said first and second time periods imaged by the camera are of substantially the same time duration.

33. The apparatus according to claim 31, wherein said camera includes a shutter actuated during the imagining of the second frame of each pair to produce a time duration of imaging the second frame of the pair substantially equal to that of imagining the first frame of the pair.

34. The apparatus according to claim 31, wherein the exposing of the second frame in each pair is synchronized with the readout of the first frame in the respective pair.

35. The apparatus according to claim 31, wherein said camera generates said series of frame pairs such as to have a short time interval between the frames of each pair, and a longer time interval between the pairs of frames.

36. The apparatus according to claim 19, wherein said optical detector includes a first imaging device for producing and display the image of the object, and a second imaging device for producing and displaying the enhanced image of said target portions overlayed on the image of the object.

37. The apparatus according to claim 19, wherein said optical detector includes an imagining device for imaging the object, and a filter mask assembly attached in front of said imaging device, said filter mask assembly including an RGB color filter mask interleaved with an emission (E) filter mask for separating and displaying the output of said imagining device into a color image of the object from RGB pixels, and an enhanced image of said target portions from E pixels overlayed on the image of the object.

38. The apparatus according to claim 19, wherein said optical detector includes an imagining device for imagining the object, a beamsplitter, for splitting the image of the object into at least four identical images and projecting said images onto four spatially separated quadrants of the optical sensor, and a special filter installed in front of said imagining device, the area of said imagining device being divided into at least four quadrants, one for each of the three colors RGB, and the fourth for the emission light; said RGB quadrants of the imaging device being used for synthesis of color image of the object, said E quadrant of the imaging device being used for displaying the enhanced image of said target portions.

39. The apparatus according to claim 19, wherein said control system exposes said object in said first time periods only to said ambient light, and exposes the object in said second time periods only to the excitation light.

40. The apparatus according to claim 39, wherein said time periods alternate at a high rate so as to eliminate or minimize flicker in the composite image displayed.
